(19) United States
(12) Patent Application Publication
(10) Pub. No.: US 2023/0123230 A1
(43) Pub. Date: Apr. 20, 2023

(54) SYSTEM AND METHOD FOR PHENOTYPIC CHARACTERISATION OF AGRICULTURAL CROPS

(71) Applicants: PONTIFICIA UNIVERSIDAD JAVERIANA, Bogotá (CO); CENTRO INTERNACIONAL DE AGRICULTURA TROPICAL-CIAT, Palmira (CO)

(72) Inventors: Andrés JARAMILLO BOTERO, Bogotá (CO); Hernán Darío BENÍTEZ RESTREPO, Bogotá (CO); Juan Andrés CARDOSO, Bogotá (CO); Luis Eduardo TOBÓN LLANO, Bogotá (CO); Maria Camila REBOLLEDO, Bogotá (CO)

(21) Appl. No.: 17/760,301

(22) PCT Filed: Feb. 14, 2020
(86) PCT No.: PCT/IB2020/051255

§ 371 Date: Aug. 6, 2022

(30) Foreign Application Priority Data
Feb. 7, 2020 (CO) NC2020/0001355

(51) Int. Cl. G01N 33/02; G06V 20/10; G06V 10/143

(52) U.S. Cl. CPC G01N 33/025; G06V 20/188; G06V 20/194; G06V 10/143

(57) ABSTRACT

This invention shows a system and method for phenotype characterization of agricultural crops, comprising at least one support device which can be reconfigured in an autonomous or controlled remote manner. It includes a central embedded microcontroller connected to atmospheric sensors located in the upper body, a microcontroller connected to a multi-spectral camera located at the distal end of the arm. The central microcontroller is connected to a base microcontroller that receives signals from soil sensors. A solar panel provides the energy source to a regulating unit that powers the microcontrollers. The system contains a communication unit that includes a router wirelessly connected to Internet.

### TECHNICAL FIELD

This invention is related to the technical field of phenomics, specifically, agricultural crop phenotyping. Particularly, this invention refers to a large scale high resolution system and method for multiple crop phenotyping, with data remote acquisition and processing using a large variety of sensors.

### OBJECT OF THE INVENTION

Provide a method and/or system for real time characterization of phenotypical traits of crops, and its corresponding correlation analysis with the genotype, by means of data remote acquisition and processing using sensors in a synchronic and asynchronous manner. The method and/or system allows to identify the response of the plant to biotic and abiotic stress, and facilitate the development and improvement of agricultural varieties with higher tolerance to such stress.

### STATE OF THE TECHNIQUE

Phenomics focuses in measuring different traits of certain organisms, at different ontogenetic levels and in diverse environments. This involves the application and development of tools to discover such traits.

When developing crops, farmers use phenomics to meet the increasing demand for stable and flexible varieties. The progress in the application of a phenomic approach in large reproductive populations or diversity panels has faced three challenges that pose a setback: 1) Restrictions in the capacity for phenotyping the field and environmental characterization, 2) The cost of conventional technologies such as person-to-person and person-computer communication; and 3) The analysis of collected data.

Non-destructive high precision phenotyping techniques have become very attractive as an efficient way to elucidate the genetic expression of crops in specific environments.

High-performance phenotyping technologies are used to grow plants and for precision agriculture, to figure out key qualitative traits involved in the response of plants to biotic and abiotic stress, that would facilitate the screening process to develop new and improved varieties of plants for different field conditions.

Patent application US 2017/032544 shows a computer implemented method, that comprises the following: receiving in one or more computer devices, one or more specifications and one or more resource limitations for a crop being monitored; generating in one or more computer devices one simulation using an automatic learning algorithm to determine whether one or more specifications and one or more restrictions result in a growing solution for the crop. The simulation is limited even more by the historical data on one or more variables that affect crop production: receiving in one or more computer devices a modification of at least one restriction after determining that the simulation does not generate a crop solution for the crop; and executing in one or more computer devices the simulation to predict the growth of the crop at specific time intervals of one crop cycle for the crop after the simulation that generates a crop solution for the crop.

Patent U.S. Pat. No. 5,253,302 shows an automatic optic sorting method for plants that comprises the following steps: (a) obtain an image of a plant with a color video camera; (b) pixel digitization of color video signals obtained from the color video camera; (c) pixel sorting of color video signals digitalized in accordance with the pre-determined color types, where such types of color comprise sets of color vectors randomly arranged which are attributed a specific type code under supervised learning on the basis that they pertain to the same significant regions; (d) segment the image of the plant to obtain a background image and the images of different parts of the plant based on the assignment of the stored image pixels to the predetermined color types; (e) determine at least one geometrical feature and color feature based on at least one segmented image of the plant and the segmented images of the parts of the plant; and (f) at least evaluate one of the characteristics determined in the form and the color features to establish a quality measure.

Patent U.S. Pat. No. 6,009,186 shows a method to harvest agricultural crop material by using a machine; this method comprises the following steps: determine a proportion of at least one product in a mixture of products that contain fruits and foreign bodies following these secondary steps: make photos using a close infrared device to obtain at least an initial image of the mixture to be examined; thresholding that comprises assigning to each pixel of the image, one of every two levels of extreme grays, corresponding to the initial level of gray of the pixel related to a specific threshold; and calculate the proportion that comprises a registry, in at least one area of the image produced by the threshold, the number of pixels of at least one level of extreme gray to determine a proportion of the area between areas respectively occupied by the crop material and foreign bodies in the image; and using the determined proportion to adapt at least one operative parameter of the machine that has an effect on such proportion.

In regards to the mentioned state of the technique, this invention resolves the issue related to phenotype characterization of agricultural crops, providing a system that integrates a structural component and an electronic and logical support component to measure and transmit real time data on the soil, the atmosphere and plant variables to geo-referenced stations.

### DESCRIPTION OF THE INVENTION

This invention originates from a response to the need to solve different problems related to phenotyping methods of large agricultural crops and diversity panels offering a system that integrates real-time data processing and transmission of soil, atmosphere and plants variables to fixed geo-referenced stations using cloud storage, and a boosting database engine for web analysis and visualization, that captures phenotypical data from both fixed and mobile land and air distributed sources.

The description of this invention is not intended to limit its scope but to be used as a specific example of such invention. A qualified person in this area will understand that the equivalent modalities are no separated from the spirit and scope of this invention in its broadest form.

To have a better comprehension of this invention, please find below a detailed explanation of certain technical terms used in the description.

Within the context of this invention, “phenotyped” means the characterization of the genetic expression of crops in specific environments.

This invention shows a system for agricultural crop phenotyping, that has three components: a mechanical-structural component, an electronic component and a logical support component.

The system of this invention integrates such components in order to characterize crops in a non-constructive way with measurable variables related to the soil, the atmosphere and the plants in order to increase the capacity of the team in charge of carrying out the studies or genetic identification that will allow them to include great extensions of agricultural crops.

This invention solves the problem derived from restrictions related to field phenotyping capacity and environmental characterization; and also the costs related to conventional technologies such as person-to-person communications and person-computer communications; and the analysis of collected data.

The system object of the present invention measures soil, plant and atmosphere variables, using dedicated sensors; such variables are processed by embedded microcontrollers. The status of the variables measured are coordinated by the central microcontroller that contains a Wi-Fi module, a 3G module and a LoRa module which is a wide area network and low power technology. Likewise, the system comprises a low-cost camera with a multispectral filter to capture images that are processed in situ to calculate the different indexes/risks of the plant.

### DETAILED DESCRIPTION OF THE INVENTION

FIGS. 1 thru 6 show a support device functioning as the structural device of the system. The support device is divided into four parts: an anchor body (10) attached to a lower body (20) that is attached to an intermediate telescopic body (30) and the latter is attached to an upper body (40) with two of rotational degrees of freedom (waist and shoulder).

FIG. 1 is an isometric view of the support device. FIG. 2 is a front view, and FIG. 3 a lateral view.

The anchor body (10) has an axle-shape stem (11) that forms or supports and anchor device such as a threat or propeller (12), in order to fix the support device to the soil in a stable manner. The anchor body (10) may have other fixtures such as flanges or supports to attach it to a fixed or mobile base.

The lower body (20) contains a central processing unit to manipulate soil measurements, a rechargeable battery powered by solar panels and soil sensor fixtures. This module can work as a stand-alone unit, and the central processing unit for wireless status communication would be located in the upper part. This lower body (20) also contains a hollow cavity that is a volume control chamber (21) to measure greenhouse gases. Methane or nitrous oxide sensors may be located in this chamber.

The intermediate body (30) contains a retractile telescope that extends the height of the upper body up to 2 meters and is adjusted in real time to the growth of the crops. This mechanism allows to maintain the level of resolution and minimize variances in the measures of the plant images, as well as maximize the capture of photons in the photovoltaic panels.

The upper body (40) contains a wireless communication unit (that may be installed in the lower body for stand-alone operation), atmospheric variable sensors and an arm (41) with multispectral camera with end effector (210) to calculate the normalized difference vegetation index of a crop. A common bus allows the communication of all the parts.

FIG. 4 shows a support device where the intermediate body (30) has a translational degree of freedom throughout the Z reference edge executed by the intermediate body (30), one rotational degree of freedom around the Z reference edge executed by the upper body (40). The multi-spectral camera is located at the distal end of the arm (41). The arm has one rotational degree of freedom (41) to maximize the exposure to radiation of the photovoltaic panels, allow capturing images at different points of the crop, and minimize the profile of the system under conditions of extreme wind due to automatic retraction when the wind speed exceeds 60 km/h.

FIG. 5 shows the support device in an upper view that shows the rotational degree of freedom of the upper body (40).

FIG. 6 shows a lower isometric view of the support device, and shows a lower body (20), the intermediate body (30) and the upper body (40).

FIG. 7 associated to FIGS. 1 thru 6 shows the phenotype characterization system, that comprises at least one support device that includes a central microcontroller (100) that can be embedded and connected to atmospheric sensors (110) located in the upper body (40), where the camera microcontroller (200) obtains a signal from the multi-spectral camera (201) located at the distal end of the arm (41); the central microcontroller is connected to a base microcontroller (300) that obtains signals from the soil (310); the central microcontroller (100) is powered by the regulating unit (400) that is fed by a solar panel (500); the phenotype characterization system also has a communication unit (600) that includes a router (610) connected to internet (620) and to the cloud.

The intermediate bodies (30) and the upper body (40) are attached to a drive unit (320) that includes a motor controller (329) and the elevation motors (321) attached to the intermediate body (30) and the rotating motor (322) and the arm motor (323) attached to the upper body (40).

The upper body (40) of the structure that comprises the structural component of the system measures the atmospheric variables by means of atmospheric sensors (110): speed and wind direction, relative humidity and air temperature, air concentration of methane.

The atmospheric sensors (110) may include, in a partial, combined or complete manner selected sensors, such as: speed sensor and wind direction sensor (111), relative humidity sensor (112), temperature sensor (113), concentration of methane sensor (114), radiation sensor (115) and other sensors.

In a particular modality, the system uses a QS-FS wind speed sensor with 0.2 m/s as initial threshold and a 1 m/s precision. This unit has an output voltage between 12 and 24 V converted into digital form with and ADC.

The system uses a Modbus standard communication protocol to transmit these data to the processing center in the upper body of the structural component of the system. This is complemented with a wind direction unit that delivers between 0 and 12 V proportionally to the relative orientation.

In a particular modality, the system uses a sensor to measure the relative humidity and atmospheric temperature. The relative humidity varies from 0 to 100% RH with a 4.5% precision and a 0.1% RH precision, and eight second (8 second) response time. Again, this is an analog sensor, therefore its signal is converted into digital with a resolution of 12 bits, 0.05% RH/bit. It can operate from −40° to 124° C. with a 0.5° C. precision and a resolution of 0.1° C. It has a 30 second response time.

In a particular modality, the system uses a sensor to measure the solar radiation level. It has a sensitivity of 0.2 mV per μmol/mss, a calibration factor of 5.0 μmol/mss per mV (reciprocal of sensitivity), with a caliper uncertainty of ±5%, a repeatability lower than 1%, with a drift lower than 2% per year and non-linearity lower than 1% (up to 4000 μmol/mms).

In a particular modality, the system uses a CH4 methane sensor that detects concentrations between 300 and 10000 parts per million (ppm) in the free air, and its purpose is to attain a differential measure between the methane atmospheric sensor (114) with respect to the methane sensor measure (314) in the volume control chamber (21) of the lower body (20). It has an analog output, functioning with +5.0 volts and consuming approximately 150 milliamps.

In addition, the lower part of the support device has at least four soil sensors (310) installed on the soil that capture data from the pH (311), humidity (312), and temperature (313) variables, and a methane concentration sensor (314) located in the volume control chamber (21).

In a particular modality, the system uses a pH sensor (311), that measures a 0 to 14 range with a linear output voltage between 0 to 5 V, with a +/−0.1 pH error.

In a particular modality, the system uses a pH sensor (311) that requires pre-calibration with a reference fluid. The unit consumes 10 mA, has a response time of 60 seconds and operates with a relative humidity of 95% and a nominal relative humidity of 65%.

In this particular modality, the system has a zero reference point in 7.0±0.5 pH and offers an alkaline error of 0.2 pH. The working temperature of this system ranges between −10 and 50° C., and again its output analog signal between −400 and 400 mV requires also an ADC converter.

In a particular modality, the system uses a humidity sensor (312) specified as a capacitive sensor that operates at +5.0 volts at 10 mA, with a response time of 10 ms to determine the humidity of the soil. It uses a sensor to measure the humidity of the soil, delivering an output of 0-3 volts with a precision of 2%. This signal is converted into a digital signal with a 14 bit ADC converter.

In a particular modality, the system uses a temperature sensor (313) with a range between −55 and 125° C. with a precision of 0.5° C., to measure the temperature of the soil. The system requires a 12 bit ADC and a 12C connection, at a 750 ms rate per conversion.

In a particular modality, the system uses a methane CH4 sensor (314) that detects concentrations between 300 and 10.000 ppm, in the controlled volume of the lower body (which is intended to obtain a differential measure with respect to the methane level in free air in the upper module). It has an analog output, works with +5.0 volts and consumes 150 milliamps.

The regulation unit (400) has a regulator (410) connected to an engine regulator (430) and a microcontroller regulator (440); the regulator (410) is connected to a battery (420); the regulation unit (400) is powered by a solar panel (500).

The energy source comprises a solar panel (500) that has a voltage variation, and uses a regulator to ensure a fixed voltage all the time. The voltage provided by the regulator charges the battery. The regulator provides the voltage to charge the battery, which in turn feeds two regulators, one regulator for the engine tension and another regulator for the microcontroller tension. These two regulators are activated depending on whether variables are being sensed or a signal is sent to the actuators (engines).

In a particular modality, the upper body (40) of the system support device integrates a camera (210) at the end of an arm rotated by gravity (41) to capture NIR, multi-spectral, thermal and RGB imaging in order to collect data to calculate the normalized difference vegetation index (NDVI) of plants, and also the nitrogen levels of leafs. This information is processed in an embedded micro-processing unit, or an embedded microcontroller. This embedded processing unit refers and includes base plates or reduced plates.

In a particular modality, the camera used is a PiNIR B2 type camera.

In addition, the system has a software platform that executes the analysis of collected data. The software platform integrates the above mentioned components. The system includes the following elements and data sources:

Low-cost sensor geo-referenced fixed nodes for the soil/plants/air variables;

The mobile units (land or air, drones),

Satellite images;

A long range and low consumption distributed wireless network architecture with cloud unloading/loading capacity from the cloud, and

Processing and data analysis tools in the cloud

In a preferred modality of the invention, the measuring is carried out by each sensor with a pre-determined sampling time between 1 minute and 24 hours. Then, the microcontrollers and turned on, a timeout is provided for the stabilization of the signal, sampling of all sensors is conducted, the Wi-Fi/3G or LoRa circuit is turned on, the information of each sensor is sent, and finally, the microcontroller is turned off until the next sensing event. The wireless transmission of data takes place for later storage in a software platform (timed database).

The base microcontroller controls the movement of the body of the articulated mechanical system with actuators or motors in two ways: first, an algorithm having predetermined values and a defined periodicity activates the elevation motor causing the body to move along the vertical axle in order to increase the height proportionally to the growth of the plant, or (2) with a camera that captures images, the algorithm processes such images and sends a signal to the elevation motor to either move or retain the body on the vertical axle depending on the growth of the plant. Likewise, when the elevation drive moves up, the rotating drive captures different images.

In the preferred modality of the invention, the upper body (40) of the support device that forms the structural component, has three degrees of freedom with a translational movement from 60 to 120 cm in the intermediate body, a rotational movement between 180 and 360 degrees and a rotational movement between 0 and 180 degrees in the arm. All the axles are driven by DC motors without brushes, or permanent magnet stepper motors, fed by electric power from a solar energy source.

The electronic component of the system comprises a group of microcontrollers fed by electric power from a solar energy source. Such microcontrollers carry out the sensing operation, the wireless communications and control the actuators connected to a cloud hosted service.

The system provides an analysis of digital images obtained by NIR, multispectral, thermal and RGB infrared cameras, to calculate the normalized difference vegetation index (NDVI) in agricultural soils using a high precision low-cost proximal detection platform.

In addition, the system comprises a logical support based on a microcontroller operative system that drives the electric mechanisms and components, and also, based on a web portal to extract, store, monitor, sort and process data in real time.

The field experiments show that the values from NDVI are highly correlated to the NDVI measures provided by an ASD-FieldSpec 2 professional spectrum-radiometer that operates within a range of 325-1075 nm.

FIG. 8 shows the work flow and the NDVI data for a crop, for example Brachiaria. It compares the data obtained with an NDVI commercial device, finding a directly proportional ratio between the NDVI spectrum as a function of the NDVI of the present invention.

The system uses an empiric method of on-line calibration (ELCM) for radiometric setting of the camera, as shown in FIG. 8.

The logical support component of the system includes a portal with a specific graphic user interface (GUI) for users and farmers in general.

The back-end is structured as follows:

An API for mapping (with navigation and object geo-referencing in the map):

Google Earth, a back-end CMS that users may manipulate in natural language and through a standard navigator, with support for different languages: WordPress, Joomla, Drupal.

A database design for temporary storage of status variables: MySQL. This database is used for real-time data collection.

Manageable web services: SOA, SOAP.

Programming capacity and incorporation of processing scripts and Python based-analysis.

Support for mobile platforms: ISO or Android.

The database structure referring to the sensing sources, contains the following tables:

Region (contains: country, sub-region, farmer—alphanumeric)

Crops (types of crops: grains, rice, Bracharia, cassava, cane, others to be defined—alphanumeric)

Genotypes per crop (contains alphanumeric code and description, for example G1, G2, G3 . . . Gn).

Networks (stations: name, region, sub-region, satellite, mobile or fixed—alphanumeric)

Each network definition contains support for each of the following elements:

Satellite station (satellite images in specific areas and contain: satellite name, origin, region, date and time).

Mobile stations (contains: name, sub-region, crop, flight autonomy, timed data path as crossing points—or geographic coordinates, 2D and 3D images). The user is able to select the path recorded that has to follow a drone within a specific time.

Fixed stations (1, 2, n) containing: name, sub-region, crop, coordinates and estimates information:

a) Weather (, rain, wind direction and speed, temperature, relative humidity, CO2, CH4, radiation). It is possible to add sensing components, such as labels associated to the type of data provided subsequently.

b) Plants (vegetation numerical index, 2D and 3D images, videos . . . ) It is possible to add new labels associated to type of data provided later.

c) Soil (temperature, relative humidity, N2, pH, water content, gravimetric content . . . ). It is possible to add new labels associated to type of data provide later.

Type of expected data: numerical (integer numeric data, float numeric data), images (raw images, JPG, PNG), video (MP4).

The system presented in this invention allows to identify relationships between phenotype and genotype

The system related to this invention provided a phenotype characterization for agricultural crop that comprises the following stages:

a. Use a solar panel (500) as energy source for the system and to power to the regulation unit (400).

b. Activate the sensors (110) (210) 310).

c. Obtain data from the sensors (110) 310).

The sensors obtain signals from the soil and the atmosphere; the atmospheric sensors (110) include wind speed and direction sensors, relative humidity sensors, temperature sensors, methane concentration sensors, radiation sensors, among others; and soil sensors (310) provide data on pH, humidity and temperature.

d. Adjust the position of the support device with a driving unit (320) that includes a motor controller (329) and the elevation motors (321) associated to the intermediate body (30) and the rotating motor (322); and the arm motor (323) associated to the upper body (40).

e. Radiometric calibration of the chamber (210).

f. Obtain images with a multi-spectral camera (210).

g. Transmit the data obtained using a communication unit (600) that includes a router (610).

h. Stop and hibernate and return to stage b.

i. Process the images and data obtained by the sensors (110) (210) (310).

j. Calculate the NDVI to characterize the phenotype in relation to the genotype.

Stage c includes a differential measuring in ppm between the methane sensor (114) and the methane sensor (314) contained in the volume control chamber (21).

FIG. 9 shows a screen shot of the user graphic interface (GUI) of the system for phenotype characterization.

The way in which this invention has been described, shows a system and method for phenotype characterization of agricultural crop, that includes three components: structural, electronic, logical support. This solves the issue related to restrictions in the field phenotyping capacity, and environmental characterization; the costs of conventional technologies such as person-to-person communication and person-computer communication.

The terms “upper, lateral, front, lower” refer to the relative position of the elements with respect to a normal location on the land surface. It is also related to the technical drawing reference system ISO E.

Although this invention has been described above, it is clear that the modifications and variations that maintain the spirit and scope of this invention will be within the scope of the attached claims.

### DESCRIPTION OF FIGURES

In order to complement the description and for a better comprehension of the technical characteristics of the invention, the following figures accompany this descriptive memory and is part thereof:

FIG. 1. General isometric view of the support device

FIG. 2. Front view of the support device

FIG. 3. Lateral view of the support device

FIG. 4. Front view of the support device.

FIG. 5. Upper view of the support device

FIG. 6. General isometric view of the support device

FIG. 7. Overview of the system for characterization of agricultural crop phenotyping

FIG. 8. Image and comparison processing system

FIG. 9. Screenshot of the system graphic interface.

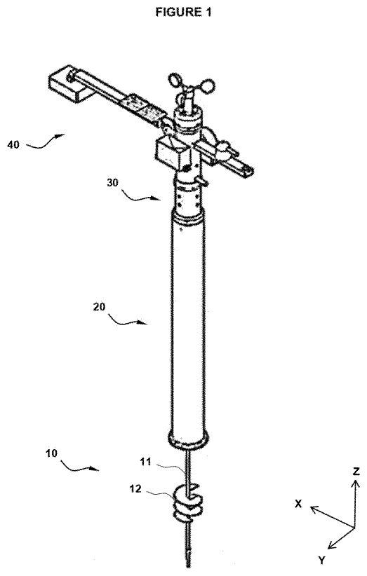

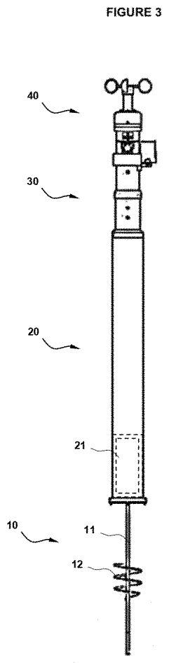

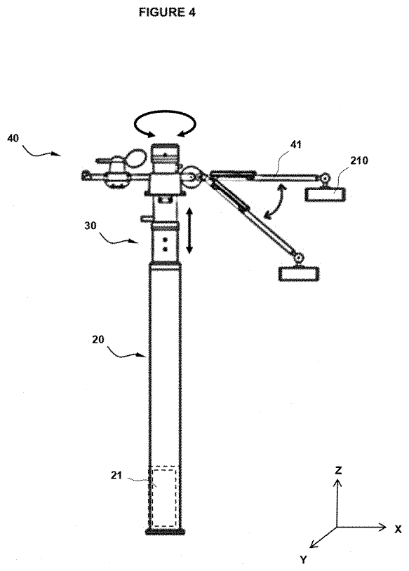

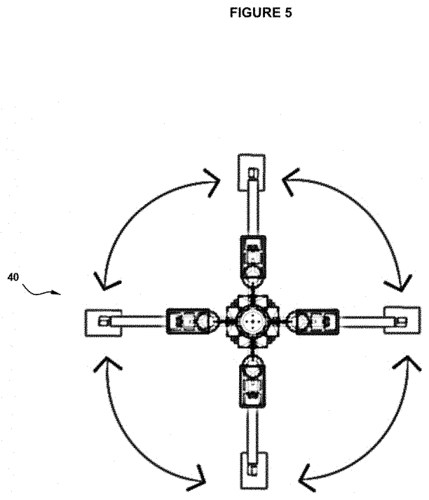

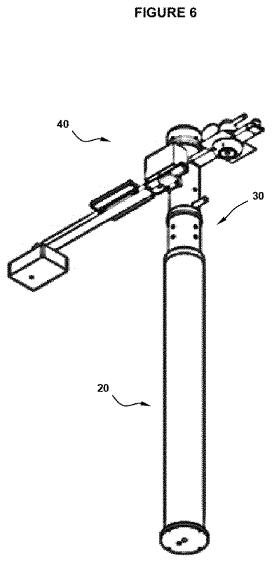

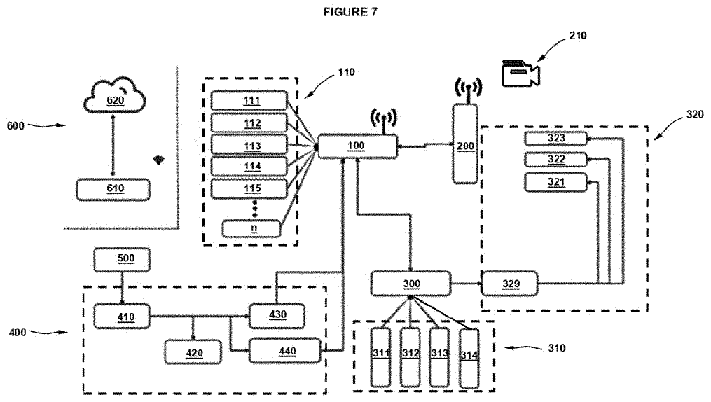

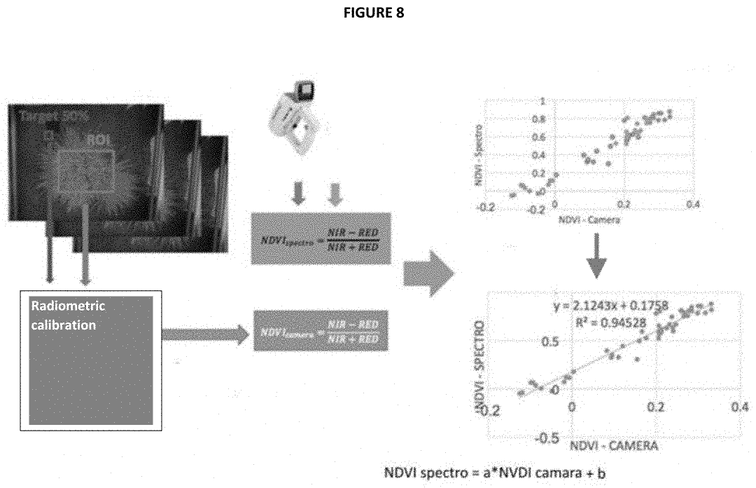

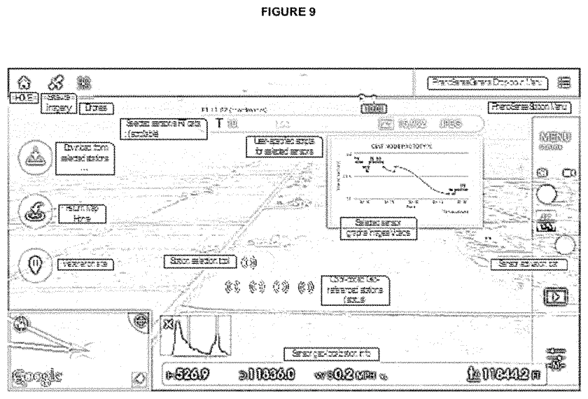

1. A system for phenotype characterization of agricultural crops featuring at least one support device that includes a central embedded microcontroller (100) connected to atmospheric sensors (110) located in the upper body (40). One embedded controller (200) receives a signal from the multi-spectral camera (210) located on the distal end of the arm (41); a central microcontroller (100) connected to a base microcontroller (300) obtains signals from the soil sensors (310). The microcontrollers, sensors and actuators are powered by the regulating unit (400) that is fed by a solar panel (500) as energy source. The system, also has a communication unit (600) that includes a router (610) with a wireless connection to Internet.

2. In accordance with claim 1, the support device includes a lower body (20) attached to an intermediate body (30) that has one translation degree of freedom; and this intermediate body (30) is attached to the upper body (40) with two rotational degrees of freedom.

3. In accordance with claim 2, the lower body (20) also has a volume control chamber (21).

4. In accordance with claim 1, the atmospheric sensors (110) are selected among: a wind speed and direction sensor (111), a relative humidity sensor (112), a temperature sensor (113), a methane concentration sensor (114) and/or a radiation sensor (115).

5. In accordance with claim 1, the soil sensors (310) include a pH sensor (311), a humidity sensor (312), a temperature sensor (313) and a methane sensor (314).

6. In accordance with claim 5 the methane sensor (314) is located in a volume control chamber (21).

7. In accordance with claim 1, the upper body (40) has an arm (41) with a a one rotational degree of freedom.

8. In accordance with claim 1, the intermediate body (30) is a telescope type body, to minimize the effects of the positional variance of the sensors during the growth of the plant.

9. In accordance with claim 1, the support device also has an anchor body (10).

10. In accordance with claim 9, the anchor body (10) includes an axle-shaped stem (11) that forms or fixes an anchor mechanism such as threat or propeller (12).

11. In accordance with claim 1, the multispectral camera (210) captures NIR imaging, multispectral imaging, thermal imaging and RGB imaging.

12. In accordance with claim 1, the system also includes a logical support with a portal graphical user interface that deploys and sorts information in real time about regions, parcels, varieties among others.

13. A method for phenotype characterization of agricultural crops that comprises the following stages:
Power the system by a solar panel energy source (500) and transmit such energy to a regulation unit (400);
Activate the sensors (110) (210) (310);
Obtain data from the sensors (110) (310); the sensors receive soil and atmosphere signals; the atmospheric sensors (110) provide data on wind speed and direction, relative humidity, temperature, methane concentration, and radiation; the soil sensors (310) provide data on the pH, relative humidity, temperature, methane and nitrous oxide;
Adjust the position of the support device, by a drive unit (320) that includes a motor controller (329) and the elevation motors (321) attached to the intermediate body (30) and the rotating motor (322) and an arm motor (323) attached to the upper body (40);
Radiometric camera calibration (210);
Obtain the images with the multispectral camera (210);
Transmit the data obtained using a communication unit (600) that includes a router (610),
Stop and hibernate and return to the sensor activation stage (110) (210) (310);
Process the images and data provided by the sensors (110) (210) (310),
Calculate the NDVI and characterize the phenotype.

14. In accordance with claim 13, during the stage of data collection by the sensors (110) (310) a differential measurement in ppm is performed between the methane sensor (114) and the methane sensor (314) contained in the volume control chamber (21).
